(19) United States
(12) Patent Application Publication
(10) Pub. No.: US 2006/0067997 A1
(43) Pub. Date: Mar. 30, 2006

(54) Phospholipid complexes of lexitropsins, their preparation and use in therapeutic formulations

(76) Inventors: Federico Arcamone, Nerviano (IT); Ugo Raffaello Citernesi, Arcore (IT)

Correspondence Address: MATHEWS, SHEPHERD, MCKAY, & BRUNEAU, P.A., 100 THANET CIRCLE, SUITE 306, PRINCETON, NJ, 08540, US

(21) Appl. No.: 10/519,889

(22) PCT Filed: Jun. 26, 2003
(86) PCT No.: PCT/EP03/06745

§ 371 Date: Sep. 20, 2005

(30) Foreign Application Priority Data
Jul. 2, 2002 (IT) MI2002A001455

(51) Int. Cl. A61K 38/06; A61K 38/05; A61K 9/127

(52) U.S. Cl. 424/450; 514/18

(57) ABSTRACT

Pharmaceutical formulations constituted by a lexitropsin phospholipidic complex in the form of liposomes, micelles or nanoparticles exhibit optimal pharmacological properties in respect to other formulations containing active principles belonging to the same chemical class in both the topical or parenteral treatment of infectious and/or tumour diseases.

### SUMMARY OF THE INVENTION

The present invention refers to pharmaceutical formulations constituted by a phospholipidic phase containing a lexitropsin and to their use for the treatment of local or general infections as well as tumour diseases in humans and in animals. Such formulations exhibit optimal pharmacological properties in respect to other, both topical and parenteral, formulations of antiinfective and antitumour agents within the class of the lexitropsins.

Object of the present invention are therefore the preparation and the therapeutic use of formulations based on liposomes, micellar aggregates or more generally phospholipidic complexes containing a lexitropsin of general structure I

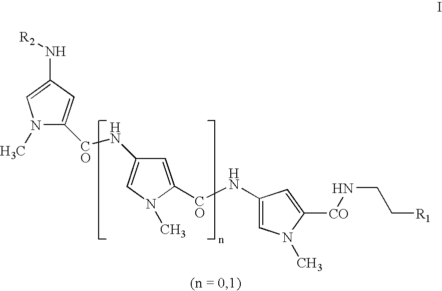

in which R₁ is a functional group, preferably a basic one such as a simple or substituted amidine, a secondary or tertiary amine, a quaternary ammonium group, a simple or substituted guanidine, examples of which, without limiting the present invention, may be

—C(NH)NH₂, —C(NH)NHR₃, —NH₂, NHR₃—N(R₃)₂, —NR₃R₄, —NH—C(NH)NH₂, —NH—C(NH)NHR₃, —N(CH₂)₄, —N(R₃)₃+

whereas R₂ represents an aliphatic, aromatic, or arylaliphatic acylic group, also if substituted with atomic groups containing one or more heteroatoms such as atoms of oxygen, nitrogen, or R₂ represents a sequence of one or more residues of 1-methyl-4-aminopyrrole-2-carboxylic acid, acylated or not acylated at the N-terminus, also terminating with a residue of 1-methyl-4-carboxamidopyrrole-2-carboxylic acid or with a residue of analogue amino acids derived from an heterocycle different from pyrrole such as, without limiting the present invention, furan, imidazole, thiophene, thiazole, or derived from benzene, pyridine, a diazine, pyrimidine, substituted or not at the terminal amino group with an acylic group, or containing, in place of the free or substituted amino group a carboxamido group, and R₃ or R₄ are equal or different lower alkyl groups C₁ to C₄.

### FIELD OF INVENTION

In the therapy of many human and animal diseases the need of further improvements in the available remedies is highly desirable in view of obtaining new medical treatments endowed with higher efficacy in the absence of unwanted side effects. This is particularly urgent in the case of viral and cancer diseases and in different parasitic diseases, among which malaria is of the greatest importance because of the high number of victims it causes in underdeveloped countries (and not only in these), as well as in the case of many bacterial infections because of the development of resistance phenomena and of the reduced immune defence mechanisms in patients with AIDS or undergoing anticancer chemotherapy.

The availability of active principle endowed with potential efficacy is however not always sufficient without the solution of therapeutic problems related with their stability, bio-availability and/or tissue distribution. Therefore the need arises of appropriate, albeit not yet available, active formulations. This is the case with the lexitropsins, a class of chemical substances displaying potentially useful activity in severe health conditions due to viral infections, tumour development, protozoarian and bacterial infections.

In the following text we disclose an original solution of the problem related with the bio-availability of these substances that, according to our invention, are employed in the form of phospholipid complexes, preferably constituted by a liposomal system. Liposomes are discrete particles formed spontaneously when amphiphatic lipids are dispersed in excess water (Liposome Methodology, vol. 107, Leserman & Barbet Eds. INSERM Publications, Paris, 1982). The lipid molecules arrange themselves by exposing their polar head groups towards the aqueous phase while the hydrophobic hydrocarbon moieties stick together thus giving rise to bilayers which eventually take the form of multiple concentric spheres retaining an internal aqueous phase separated from the rest of the solution (multilamellar liposomes).

Lexitropsins are defined as compounds characterized by the presence of the residue of 1-methyl-4-aminopyrrole-2-carboxylic acid as the monomeric unit in a linear peptide type structure generally displaying a basic group such as an amidine or a substituted amine or guanidine at the C-terminal position, and a variety of acyl moieties or a carboxamide group at the N-terminal position. The lexitropsins may be microbial products or synthetic analogues of the same. In this latter case, in the place of a residue of 1-methyl-4-aminopyrrole-2-carboxylic acid one may find a similar derivative containing however a different heterocyclic or aromatic ring. Some example of known lexitropsin structures are presented in the following (formulas II-VIII a-i). Lexitropsins are endowed with interesting and useful pharmacological properties: within the group of known compounds we find exhibition of antiviral, antitumour, antiprotozoarian and antibacterial activities.

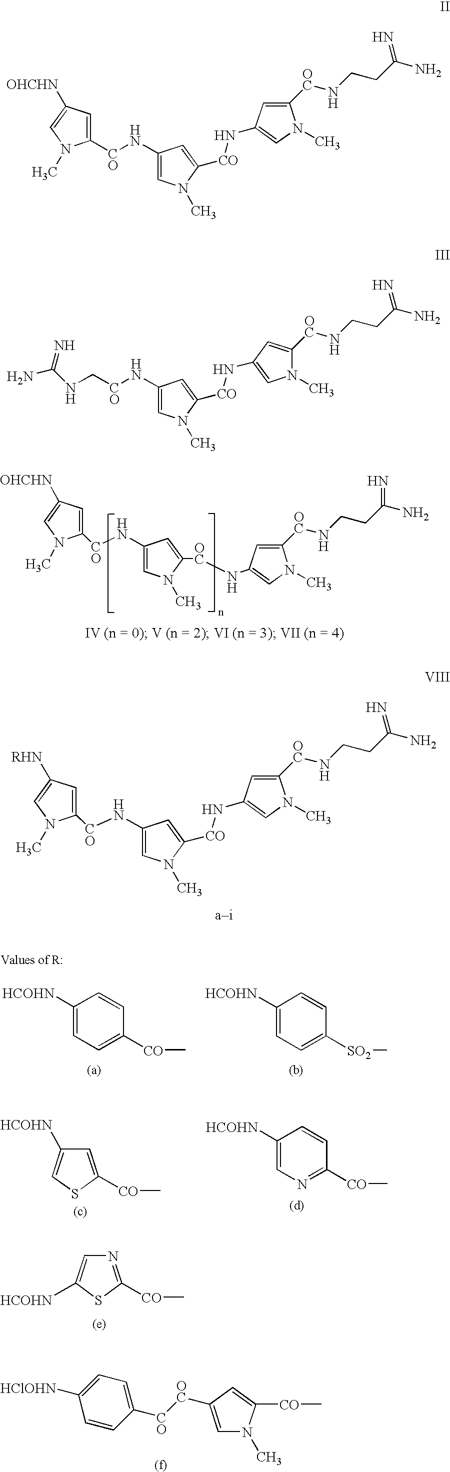

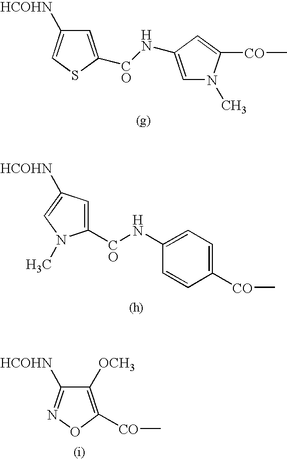

### PREVIOUS ART

Scientific literature on lexitropsins is today quite wide, but no publication or patent specifically claiming or describing liposomal or micellar formulations or, more generally, formulations based on the formation of phospholipidic complexes of lexitropsins have appeared to date.

As for the active principles exemplified here, they have been object of preceding inventions: Distamycin and distacin F. Arcamone et al., Ger. Offen. 1039198 (Sep. 18, 1957), CA 55, 2012f. Pyrroles, F. Arcamone et al. Belg. Pat. 666612 (Nov. 3, 1965), CA 65, 5444d Preparation of distamycin derivatives as antiviral, antitumor agents, F. Animati et al. PCT Int. Appl. WO 92 9,574 (Jun. 11, 1992), CA 117, 130993. Preparation of distamycin analogs as antiviral and antitumor agents, F. Animati et al. PCT Int. Appl. WO 92 14.707 (Sep. 3, 1992). CA 118, 38687; Preparation of Distamycin A derivatives as antimalarials. F. Animati et al., PCT Int. Appl. WO 94 25,436 (Nov. 10, 1994), CA 122, 105530.

The same compounds exemplified here are reported in scientific publications: Distamycin A. I. Isolation and structure of the antiviral agent distamycin A, F. Arcamone et al. Gazz. Chim. Ital., 97, 1097-1109 (1967); Distamycin A. II. Total synthesis, S. Penco et al., ibid., 97, 1110-1115 (1967). Distamycin A. III. Synthesis of analogs with modifications in the side chains, F. Arcamone et al., Gazz. Chim. Ital. 99, 620-631 (1969). Distamycin A. IV. Synthesis of analogs with different numbers of 1-methyl-4-aminopyrrole-2-carboxylic acid residues, F. Arcamone et al., Gazz. Chim. Ital., 99, 632-640 (1969). Synthesis, DNA binding and antiviral activity of distamycin analogs containing different heterocyclic moieties, F. Arcamone et al. Anticancer Drug Design, 1, 235-44 (1986). Biological activity and DNA sequence specificity of synthetic carbamoyl analogues of distamycin. A. Alfieri et al., Antiviral Chemistry and Chemotherapy, 8, 243-254 (1997).

### DESCRIPTION OF THE INVENTION

The present invention is related to pharmaceutical formulations constituted by a phospholipidic phase containing a lexitropsin and their use for the medical treatment of local and general infections as well as of tumour diseases in humans and in animals. Such formulations exhibit optimal pharmacological properties in respect to other formulations when used for the topical and parenteral administration of antiinfective or antitumour agents belonging to the class of the lexitropsins.

Typical objects of the present invention are liposomal or micellar preparations or phospholipidic complexes of distamycin (also known as stallimycin, formula II), netropsin (formula III) an analogue thereof (formulas IV-VIII a-i), or other compounds, such as IX, X and any analogue included in the general structure I useful for the preparation of medical prescriptions against infectious or cancer diseases in humans and in animals.

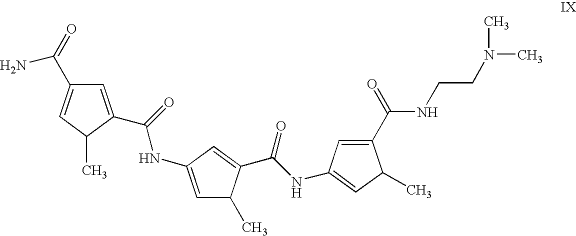

According to the present invention the pharmacological activity of every bioactive lexitropsin, being lexitropsin a chemical compound as defined above, is markedly improved by the phospholipidic formulation disclosed here and the latter can be employed in the medical treatment of local and general infections due to responsive pathogens and of cancer. An additional series of lexitropsin derivatives utilized within the scope of the present invention are the bis-amidine analogues of general structure XI (n=1-4) that are endowed with interesting antiviral and antibacterial activity and whose therapeutic efficacy could also be greatly improved by the use of the liposomal formulations described in the present invention.

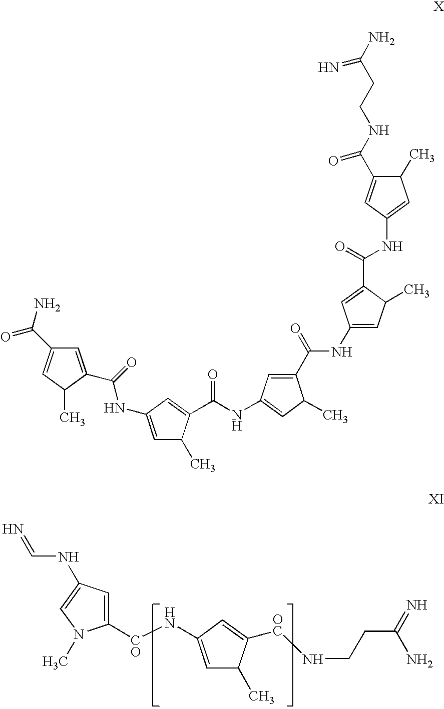

A typical embodiment of the present invention is the preparation of multilamellar liposomes, composed of phosphatidyl glycerol (PG), phosphatidyl choline (PC) and cholesterol (C) containing an entrapped lexitropsin in an amount comprised in the range 1-10 percent of the mass of the liposome. Another typical embodiment of the present invention is the preparation of phospholipidic vesicles composed by polyethylene glycol ethanolamine (PEGPE), PG and partially hydrogenated egg phosphatidyl choline (PHEPC) containing 1-10% by weight of a lexitropsin. A further typical embodiment of the present invention is the preparation of liposomes and phospholipidic complexes containing a lexitropsin in sterile and apyrogenic form. A preferred object of the present invention are liposomal formulations as described above entrapping specifically a lexitropsin of general formula I.

An important embodiment of the present invention is represented by the topical use in localized viral or tumour diseases of liposomal or micellar preparations or of phospholipidic complexes of distamycin II, or of an analogue thereof such as for instance a compound of structures III, IV, V, VI, VII, VIII, IX, X, XI, in the understanding that the invention is not limited to the use of the specific compounds indicated herein. A further important embodiment of the present invention is also the therapeutic use of sterile and apyrogenic liposomal or micellar preparation, or of phospholipidic complexes containing a lexitropsin of general formula I for the treatment by a parenteral route of local or generalized infections as well as of cancer in humans and in animals. Such formulations allow the exhibition of optimal pharmacological properties of anti-infective or anti-tumour agents belonging to the chemical class of the lexitropsins. In consequence a preferred embodiment is the parenteral use of liposomes entrapping a compound of general formula I, as for example compounds II-XI, for the treatment of viral diseases. Compound X is known as endowed of marked antimalarial activity. This property is significantly enhanced when the compound is used in the formulation as described above and can therefore be employed for the treatment of malaria in man.

A further object of the present invention is represented by the use of the above mentioned formulations for the production of pharmaceutical preparations containing them.

### EXAMPLES

### Example 1

Multilamellar liposomes are prepared by dissolving 60 micromoles of the mixture PG:PC:cholesterol (in chloroform) in the molar ratios 1:4:4 and 3 micromoles of a lexitropsin of general formula I, as an organic or inorganic acid salt (preferably the hydrochloride) in methanol, the mixture being then evaporated at room temperature in a rotary evaporator under vacuum. To the residue is then added at 37° physiological solution buffered at pH 7.4 with Tris.HCl (10 ml) and the resulting suspension is stirred at 37° overnight. The obtained heterogeneous suspension of multilamellar liposomes is extruded through a porous filter (0.2-0.4 micron) under nitrogen at a pressure of 40-80 psi at room temperature, then centrifuged at 130000×g for one hour in order to remove the non-entrapped lexitropsin. Liposomes so obtained are taken up as a suspension in 1 ml of physiological solution, dialyzed against 100 volumes of physiological solution at 37° C. and the resulting suspension is freeze-dried in a glass vial. The final product contains more than 65% of the starting lexitropsin.

### Example 2

A chloroform solution containing 50 mg each of phosphatidyl glycerol (PG), phosphatidyl choline (PC), cholesterol (C), is evaporated in a rotary evaporator at reduced pressure and all traces of solvent are removed with a current of nitrogen. The resulting lipid film is hydrated with 3 ml of physiological solution containing 39 micromoles of a lexitropsin of formula I as the salt of an organic or inorganic acid (preferably the hydrochloride) under stirring with a vibromixer apparatus at 30 sec. intervals and with standing in a water bath at 60° C. for the same period of time for a total time of 10 min. The obtained liposomal suspension is sonicated under nitrogen for 2 min. in a sanitation bath, then cooled in a bath of ice an water for 1 min. Liposomes so obtained are recovered by ultracentrifugation at 130,000×g for 20 min., then resuspended in water containing 10% (w/w) lactose and freeze-dried. The yield of entrapped lexitropsin is 90%.

### Example 3

Multilamellar liposomes containing entrapped a lexitropsin of general formula I, obtained as described in examples 1 and 2 are recovered, instead of using a centrifugation step, by ultrafiltration at cut-off comprised in the range 1000-5000 daltons, taken up in 10% (w/w) aqueous lactose and freeze-dried.

### Example 4

Multilamellar liposomes containing entrapped a lexitropsin of general formula I, obtained as described in example 1 or 2, are dialyzed against 100-200 ml of physiological solution at 37° then sterilized by filtration and the filtrate is distributed in sterile and pyrogen-free conditions in sterile vials and lyophilised.

### Example 5

Multilamellar liposomes containing entrapped lexitropsin of general formula I, obtained as described in example 3, are taken up in 10% aqueous lactose and sterilized by filtration. The sterile solution of the liposomes is finally distributed in sterile ant pyrogen-free conditions in sterile vials and lyophilised.

### Example 6

Vesicle forming lipids PEG-PE (polyethylen glycol phosphatidyl ethanolamine), PG, PHEPC (partially hydrogenated egg phosphatidyl choline) and cholesterol in the molar ratio 0.15:0.3:1.85:1 are dissolved in chloroform at a final total lipid concentration of 25 micromoles of phospholipid/ml. The solvent is removed under reduced pressure and the resulting dry lipid film is hydrated with a warm (60° C.) solution of 10 mM of a lexitropsin of general formula I as the salt of an organic or inorganic acid (preferably as the hydrochloride) in 0.9% aqueous NaCl. The hydration is performed using 1 ml of the aqueous solution for 50 micromoles of phospholipid with with 10 cycles of freezing and thawing using liquid nitrogen and a warm water bath. Optimal size of the liposomes is obtained by extrusion through two polycarbonate membranes, three cycles using 0.4 micron filters and three cycles using 0.2 micron filters. Final dimension of the liposomes is of the order of 0.1 micron in diameter. The liposomes so obtained are dialyzed against 50-100 volumes of a 5% aqueous lactose solution three times for a total time of 24 h. A fourth dialysis step lasting one h is finally performed against a solution of 5% lactose at pH comprised in the range 6.5-7. The non-entrapped fraction of lexitropsin is removed by treatment with a mixed bed of acid and basic ion-exchange resins followed by 5 min. low speed centrifugation. The liposomal suspension is then sterilized through filtration through a 0.45 micron membrane, lyophilised and stored at 5° C.

### Example 7

Multilamellar liposomes are prepared by dissolving 60 micromoles of the mixture PG:PC:cholesterol (in chloroform) in the molar ratios 1:4:4 and 1.5 mg of distamycin (II) as the hydrochloride in methanol. The mixture is evaporated in a round bottomed flask under reduced pressure and at room temperature. The resulting residue is taken up at 37° C. with a physiological solution buffered at pH 7.4 with tris-hydroxymethylaminomethane. HCl buffer and stirred at 37° C. overnight. The heterogeneous liposomal suspension is extruded through a 0.2-0.4 micron porous filter under nitrogen at a pressure of 40-80 psi at room temperature then ultracentrifuged at 130000×g for one h at room temperature in order to remove non-entrapped II. The liposomes so obtained are dialyzed against 100-200 volumes of physiological solution and lyophilised. The yield of entrapped distamycin is higher then 65% of the amount of the starting sample.

### Example 8

A chloroform solution containing 50 mg each of phosphatidyl glycerol (PG), phosphatidyl choline (PC), cholesterol (C), is evaporated in a rotary evaporator at reduced pressure and all traces of solvent are removed with a current of nitrogen. The resulting lipid film is hydrated with 3 ml of physiological solution containing 15 mg of distamycin (II) hydrochloride under stirring with a vibromixer apparatus for 30 sec. followed by standing in a water bath at 60° C. for the same period of time in repeated cycles for a total time of 10 min. The obtained liposomal suspension is sonicated under nitrogen for 2 min. in a sonication bath, then cooled in a bath of ice an water for 1 min. Liposomes so obtained are recovered by ultracentrifugation at 130,000×g for 20 min., then resuspended in water containing 10% (w/w) lactose and freeze-dried. The yield of entrapped distamycin is 90%.

### Example 9

Multilamellar liposomes, obtained as described in examples 7 or 8, after extrusion through the porous filter, are freed from non-entrapped distamycin and recovered by an ultrafiltration step at cut-off comprised in the range 1000-5000 Dalton instead of the ultracentrifugation step, than taken up in physiological solution and lyophilized.

### Example 10

Multilamellar liposomes are prepared by dissolving 60 micromoles of the mixture PG:PC:cholesterol (in chloroform) in the molar ratios 1:4:4 and 1.5 mg of compound X as the hydrochloride in methanol. The mixture is evaporated in a round bottomed flask under reduced pressure and at room temperature. The resulting residue is taken up at 37° C. with a physiological solution buffered at pH 7.4 with tris-hydroxymethylaminomethane. HCl buffer and stirred at 37° C. overnight. The heterogeneous liposomal suspension is extruded through a 0.2-0.4 micron porous filter under nitrogen at a pressure of 40-80 psi at room temperature then ultracentrifuged at 130000×g for one h at room temperature in order to remove non-entrapped X in the supernatant and recover the liposomal preparation in the centrifugation pellet. The liposomes so obtained are resuspended and dialyzed against 100-200 volumes of physiological solution, then distributed in sterile and pyrogen-free conditions in sterile glass vials and lyophilised. The yield of entrapped distamycin is higher then 65% of the amount of the starting sample.

### Example 11

A chloroform solution containing 50 mg each of phosphatidyl glycerol (PG), phosphatidyl choline (PC), cholesterol (C), is evaporated in a rotary evaporator at reduced pressure and all traces of solvent are removed with a current of nitrogen. The resulting lipid film is hydrated with 3 ml of physiological solution containing 30 micromoles of compound X under stirring with a vibromixer apparatus for 30 sec. followed by standing in a water bath at 60° C. for the same period of time in repeated cycles for a total time of 10 min. The obtained liposomal suspension is sonicated under nitrogen for 2 min. in a sonication bath, then cooled in a bath of ice an water for 1 min. Liposomes so obtained are recovered by ultracentrifugation at 130,000×g for 20 min., then resuspended in water containing 10% (w/w) lactose and filtered through a standard sterilization filter, distributed in sterile and pyrogen-free conditions in sterile glass vials and then freeze-dried. The yield of entrapped X is 90%.

### Example 12

Multilamellar liposomes, obtained as described in examples 10 or 11, after extrusion through the porous filter, are freed from non-entrapped X and recovered by an ultrafiltration step at cut-off comprised in the range 1000-50000 Dalton instead of the ultracentrifugation step, than taken up in physiological solution and lyophilised in sterile and pyrogen free conditions in sterile glass vials.

### Example 13

Vesicle forming lipids PEG-PE, PG, PHEPC and cholesterol in the molar ratio 0.15:0.3:1.85:1 are dissolved in chloroform at a final total lipid concentration of 25 micromoles of phospholipid/ml. The solvent is removed under reduced pressure and the resulting dry lipid film is hydrated with a warm (60° C.) solution of 10 mM of compound X as the hydrochloride in 0.9% aqueous NaCl. The hydration is performed using 1 ml of the aqueous solution for 50 micromoles of phospholipid with 10 cycles of freezing and thawing using liquid nitrogen and a warm water bath. Optimal size of the liposomes is obtained by repeated extrusion through two polycarbonate membranes, three cycles using 0.4 micron filters and three cycles using 0.2 micron filters. Final dimension of the liposomes is of the order of 0.1 micron in diameter. The liposomes so obtained are dialyzed against 50-100 volumes of a 5% aqueous lactose solution three times for a total time of 24 h. A fourth dialysis step lasting one h is finally performed against a solution of 5% lactose at pH comprised in the range 6.5 to 7. The non-entrapped fraction of lexitropsin X is removed by treatment with a mixed bed of acid and basic ion-exchange resins followed by 5 min. low speed centrifugation. The liposomal suspension is then sterilized through filtration through a 0.22 micron membrane, lyophilised and stored at 5° C.

### Example 14

Multilamellar liposomes are prepared as described in example 10 above substituting compound X with compound XI. The yield of entrapment is higher than 65%.

### Example 15

Multilamellar liposomes are prepared as described in example 11 above substituting compound X with compound XI. The yield of entrapment is 90%.

### Example 16

Multilamellar liposomes are prepared as described in example 12 above substituting compound X with compound XI.

### Example 17

Multilamellar liposomes are prepared as described in example 13 above substituting compound X with compound XI

### Example 18

A lexitropsin preparation, obtained as described in examples 1 to 17 is administered in a topical formulation onto the affected skin of the patient in such amount as to apply 5-50 mg of the active principle in each single application two-three times a day for a period of 5 to 7 consecutive days.

### Example 19

A lexitropsin preparation, obtained as described in examples 4 to 6 and 10 to 17 is administered by parenteral route, that is by intravenous, intramuscular or subcutaneous injection, to patients affected by a disease respondent to the lexitropsin medication at a dosage comprised in the range of 5 to 500 mg once a day for a period of 2 to 7 consecutive days.

1. A phospholipidic preparation consisting in a release system and a lexitropsin of general formula I 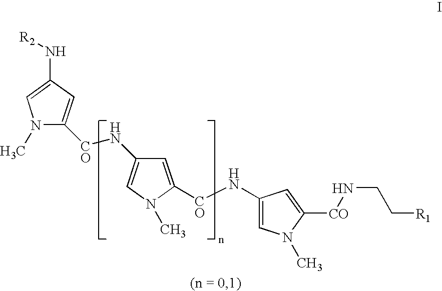
in which R₁ is a functional group, preferably a basic one such as a simple or substituted amidine, a secondary or tertiary amine, a quaternary ammonium group, a simple or substituted guanidine, selected from:
—C(NH)NH₂, —C(NH)NHR₃, —NH₂, NHR₃—N(R₃)₂, —NR₃R₄, —NH—C(NH)NH₂, —NH—C(NH)NHR₃, —N(CH₂)₄, —N₃)₃+
whereas R₂ represents an aliphatic, aromatic, or arylaliphatic acylic group, also if substituted with atomic groups containing one or more heteroatoms such as atoms of oxygen, nitrogen, or R₂ represents a sequence of one or more residues of 1-methyl-4-aminopyrrole-2-carboxylic acid, acylated or not acylated at the N-terminus, also terminating with a residue of 1-methyl-4-carboxamidopyrrole-2-carboxylic acid or with a residue of analogue aminoacids derived from an heterocycle different from pyrrole selected from furane, imidazole, thiophene, thiazole, or derived from benzene, pyridine, a diazine, pyrimidine, substituted or not at the terminal amino group with an acylic group, or containing, in place of the free or substituted amino group a carboxamido group, and R₃ or R₄ are equal or different lower alkyl groups C₁ to C₄,
the release system being a liposome, a micelle, a nanoparticle, a phospholipidic complex or a supramolecular phospholipidic structure able to incorporate a compound of general structure I in stable and reversible form.

2. A preparation according to claim 1, in form of multilamellar liposomes, composed of phosphatidyl glycerol (PG), phosphatidyl choline (PC) and cholesterol (C) containing an entrapped lexitropsin of formula I in an amount comprised in the range 1-10 percent of the mass of the liposome.

3. A preparation according to claim 1, in form of phospholipidic vescicles composed by polyethyleneglycol ethanolamine (PEGPE), PG and partially hydrogenated egg phosphatidyl choline (PHEPC) containing 1-10% by weight of a lexitropsin.

4. A preparation according to any one of claims 1-3, comprising distamycin (II) in the form of an organic or inorganic salt, preferably as the hydrochloride, as the active ingredient.

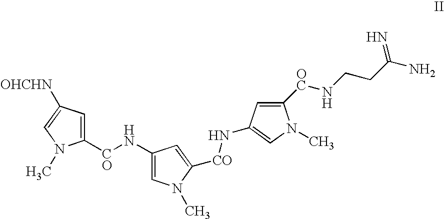

5. A preparation, according to any one of claims 1-3 comprising a compound X in the form of an organic or inorganic salt, preferably as the hydrochloride.

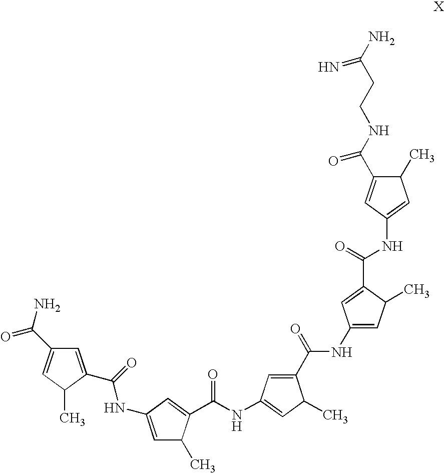

6. A topical preparation according to any one of claims 1-5, containing from 0.1 to 10% of active principle.

7. An injectable preparation according to any one of claims 1-5 providing a dosage from 0.1 to 20 mg of a lexitropsin of general formula I, II or X per kg body weight.

8. The use of the preparations of claims 1-7 for the preparation of medicaments for the treatment of vial, or bacterial, or protozoarian infections.
